(19) United States
(12) Patent Application Publication
(10) Pub. No.: US 2004/0133260 A1
(43) Pub. Date: Jul. 8, 2004

(54) Lumenal vascular compliance device and method of use

(76) Inventors: Robert S. Schwartz, Rochester, MN (US); Robert A. Van Tassel, Excelsior, MN (US)

Correspondence Address: INSKEEP INTELLECTUAL PROPERTY GROUP, INC, 1225 W. 190TH STREET, SUITE 205, GARDENA, CA, 90248, US

(21) Appl. No.: 10/665,785

(22) Filed: Sep. 17, 2003

Related U.S. Application Data
(63) Continuation-in-part of application No. 10/192,402, filed on Jul. 8, 2002.
(60) Provisional application No. 60/412,122, filed on Sep. 17, 2002.
(60) Provisional application No. 60/473,988, filed on May 28, 2003.
(60) Provisional application No. 60/303,573, filed on Jul. 6, 2001.

(51) Int. Cl. A61F 2/06

(52) U.S. Cl. 623/1.1

(57) ABSTRACT

The present invention modifies the compliance of a vascular system by providing an elastic member, capable of reducing peak pressure and blood flow from the heart. These embodiments further allow for reduction of peak systolic pressure while increasing diastolic pressure and flow. In one embodiment, the device consists of an anchoring stent, having an elastic member with a passage for blood flow. This device is implanted percutaneously into a desired vessel location. The elastic member begins to “give” when blood pressure reaches a desired level. Additionally, the spring constant of the elastic member may be externally modified to change the compliancy. By precisely modifying the properties of the elastic member, normal arterial compliancy may be restored.

This application claims priority to provisional application 60/412,122 filed on Sep. 17, 2002 entitled “Aortic Shock Absorber” and to provisional application 60/473,988 filed May 28, 2003 entitled “Aortic Shock Absorber, V.2”. This application is also a continuation-in-part patent application of co-pending U.S. application Ser. No. 10/192,402 filed Jul. 08, 2002 entitled “Anti-Arrhythmia Devices And Methods Of Use”; which itself claims benefit of provisional application 60/303,573 filed Jul. 06, 2001 entitled “Anti-Arrhythmia Ring.” The entirety of all the proceeding applications are incorporated herein by reference.

### FIELD OF INVENTION

The present invention relates to medical devices. More particularly, this invention relates to passive devices that absorb aortic blood pressure shock, restoring elasticity to cardiovascular systems.

### BACKGROUND OF THE INVENTION

Hypertension, also known as high blood pressure, can cause heart, kidney, brain and arterial damage, leading to atherosclerosis, stroke, heart attack, heart failure, and other vascular related diseases. The exact cause of hypertension is often difficult to determine, but several factors are thought to contribute to the condition, including obesity, heavy alcohol use, family history, high salt intake, diabetes, stiffening of the vascular system, and aging. Stress, low calcium intake, and resistance to insulin may also be contributing factors. Additionally, secondary forms of hypertension can occur due to certain medications, narrowing of the kidney arteries, or pregnancy.

Almost one-third of every American adult has high blood pressure, an estimated 58 million people. Of the 58 million with high blood pressure, nearly one-third are unaware of it, and almost two-thirds are unable to control it.

Hypertension has an important and common link with congestive heart failure due to both afterload increases and deleterious changes in pressure-flow relationships of the left ventricle and aorta, the loading conditions of the left ventricle.

As the aorta ages, it loses compliance, or elasticity, through wall thickening, fibrous scar formation, cellular degeneration, expansion, and elastin degradation. The aortic wall and smaller vessels undergo hypertrophy, or fibrous thickening, in response to chronically elevated blood pressures. This hypertrophy causes increased pressure rises with accelerating rates of change, creating a positive feedback process as further described below. Such effects are thought to cause damage to the arterial wall tissue, resulting in further decreased compliance. Decreased compliance causes increased systolic pressure, which in turn causes more rapid and severe vascular wall degeneration. This sequence becomes a vicious circle of feedback events that progressively deteriorate normal aortic compliance functions, increase blood pressure, and eventually degrade left ventricular systolic and diastolic function, leading to heart failure syndromes.

The normal human aorta and large capacitance vessels are only partially resistive. The pressure-flow relationship is also partially capacitive, whereby the blood flow leads pressure for pulsatile waveforms as induced by the bolus of blood injected by the heart with each cardiac cycle. As the human vessel ages, it becomes significantly stiffer with the result being a more purely resistive structure. This means that the blood pressure rises simply because of the arterial stiffness, resulting in more work per heartbeat that the heart must expend.

Peak pressure increases in non-compliant vascular systems are believed to induce stress. The amount of stress is related to several factors, including blood pressure, blood viscosity, and velocity of the blood. This stress triggers the body's injury response mechanism which subsequently interferes with the functionality of the artery.

Several studies have examined the proximal and distal thoracic aortic area and distensibility through the cardiac cycle, and found a direct relationship with exercise intolerance in elderly patients. Patients with diastolic dysfunction have higher resting heart rates and systolic blood pressure, greater left ventricular mass, aortic wall thickness and mean aortic flow velocity. Thus, poor exercise tolerance strongly correlates with reduced aortic compliance and pressure-distensibility.

Lifestyle changes such as exercise and weight loss may help reduce hypertension. In addition, medications remain a common treatment prescribed by doctors, and may include diuretics, beta-blockers, calcium channel blockers, angiotensin-converting enzyme (ACE) inhibitors, angiotensin receptor blockers, or alpha blockers. Additionally, severe hypertension is treated with potent vasodilators such as hydralazine, minoxidil, diazoxide, nitroprusside, or similar drugs.

In this regard, the following chart illustrates the results of typical ACE inhibitor therapy with the drug Enalapril:

| | Before Enalapril | After Enalapril | Change |
|---|---|---|---|
| Parameter | (mmHg) | (mmHg) | (mmHg) |
| Mean Brachial | | | |
| Pressure | | | |
| Systolic | 163 ± 15 | 155 ± 20 | −8 |
| Diastolic | 85 ± 10 | 81 ± 10 | −4 |
| Pulse Pressure | 78 ± 16 | 74 ± 20 | −4 |
| Mean | 163 ± 15 | 155 ± 20 | −8 |
| Mean Central | | | |
| Pressure | | | |
| Systolic | 164 ± 18 | 156 ± 24 | −8 |
| Pulse Pressure | 79 ± 19 | 75 ± 23 | −4 |
| Peripheral | 2172 ± 508 | 2122 ± 462 | −50 |
| Resistance | | | |
| Proximate Aortic | 0.45 ± 0.24 | 0.49 ± 0.28 | +0.04 |
| Compliance | | | |

Mitchell G F et al, Omipatrilat Reduces Pulse Pressure and Proximal Aortic Stiffness in Patients with Systolic Hypertension, Circulation 2002:105:2955-2961. Although the results of this therapy are favorable, the disadvantage is that such hypertensive patients will be on such expensive medications for life, requiring them to take one or more pills daily. Further, these medications lack the desired efficacy in some patients while additionally producing unwanted side-effects.

Accordingly, it is desired to formulate a different treatment approach that achieves the same or better results as the above-identified ACE inhibitor therapy, but avoids the associated negative aspects of it. In this regard, one such alternate is disclosed in U.S. Pat. No. 5,409,444 (Kensey) incorporated herein by reference. While such a design may produce some improvement in reducing high blood pressure, its efficacy remains limited by a number of factors including an inability to transcutaneously change compliance, poor energy conservation, an incapacity to measure and transmit pressure, an inability to start compression until a threshold pressure is reached, an inability to secure itself with inflammation induced fibrosis, and many more. These drawbacks have held the design back from widespread use in the medical community for treatment of hypertension.

Thus, a need exists for an improved medical device and method of use for absorbing aortic shock pressure, lacking the many drawbacks of the previous design in addition to the price and side effect constraints of medications.

### OBJECTS AND SUMMARY OF THE INVENTION

One object of the present invention is to provide a method and apparatus for absorbing aortic shock pressure.

Another object of the present invention is to provide a method and device for changing the velocity, volume, and/or pressure of blood flow from the left ventricle.

Yet another object of the present invention is to provide a method and apparatus for reducing the work load of the heart in a patient with congestive heart failure, hypertension, or being normotensive.

Another object of the present invention is to provide a method and device for increasing the compliance of a vascular system.

Another object of the present invention is to provide a method and device that overcomes the disadvantages of the prior art.

Another object of the present invention is to provide a device that has a pressure-volume relationship that is capacitive, thus aiding in systolic dysfunction.

The device of the present invention also allows for the treatment of diastolic heart failure/diastolic dysfunction. It has recently been recognized that increased stiffness of the aortic and great vessels may in part be responsible for dyspnea and dyspnea on exertion. Thus, inserting a device that restores or enhances aortic compliance will partially or completely relieve the dyspnea and diastolic dysfunction as etiology.

The device of the present invention also allows for treatment of orthostatic hypotension. A partial stenosis, less than 60-70%, will create little or no clinical effect at rest. As increased flow occurs with orthostatic hypotension on arising, the enhanced flow through a partial stenosis will result in a developed gradient, supporting the central blood pressure. Moreover, a major cause of orthostatic hypotension is medication. The ability to partially or completely eliminate medication with the device will also limit the orthostatic hypotension.

The present invention relates to passive medical devices that absorb aortic pressure shock, restoring elasticity to a cardiovascular system.

Specifically, the present invention modifies the compliance of a vascular system by providing an elastic member, capable of reducing peak pressure and blood flow from the heart. These embodiments further allow for reduction of peak systolic pressure while increasing diastolic pressure and flow. Additionally, these embodiments can reduce the overall workload performed by the heart. Thus, the present invention allows for improved cardiovascular system functions, enhancing a patients health.

In one embodiment, the device consists of an anchoring platform, having an elastic member with a passage for blood flow. This device is implanted percutaneously into a desired vessel location. The elastic member begins to give or create increased volume, when blood pressure reaches a desired level. Additionally, the spring constant of the elastic member may be externally modified to change the compliance. By precisely modifying the properties of the elastic member, normal arterial compliance may be restored.

In this concept, the compliance is dynamic. Greater pressure creates greater volume through an application of the Bernoulli principle. The enhanced flow results in decreased intraluminal pressure, pulling a portion of the device into the lumen as does the sail on a sailboat. Some applications of the present invention may require the phase angle to be inductive, in other words having a phase angle that allows pressure to lead flow.

The different relationships of pressure and volumes will result in different clinical features and behavior. The device also can be made to function only above the determined set point.

The device is also dose independent. That is, the device does not function to lower blood pressure at values less than the set point at which it begins functioning. Giving a blood pressure medication to a person with borderline low hypertension would act to lower the pressure further than needed.

### BRIEF DESCRIPTION OF THE DRAWINGS

FIG. 1 is a side view of one embodiment of the present invention.

FIG. 2 is an end view of the embodiment shown in FIG. 1.

FIG. 3 is a side view of one embodiment of the present invention in an aorta.

FIG. 4 is a side view of a parallel compliant embodiment of the present invention.

FIG. 5 is side view of a single entry compliant embodiment of the present invention.

FIG. 6 is a side view of an outer cuff-like embodiment of the present invention.

FIG. 7 is a side view of a percutaneous compliant grabbing embodiment of the present invention.

FIG. 8 is a side view of an internal/external compliant embodiment of the present invention.

FIG. 9 is a side view of another embodiment of the internal/external compliant device of the present invention.

FIG. 10 is an end view of a compliant vacuum chamber with springs of the present invention.

FIG. 11 is a side view of another embodiment of a compliant vacuum chamber with springs of the present invention.

FIG. 12 is a side view of multiple compliant devices used in accordance with the present invention.

FIG. 13 is a side view of a filamentous compliant embodiment of the present invention.

FIG. 14 is a sectional view of an embodiment of an elastic member of the present invention.

FIG. 15 is a closer sectional view of the embodiment of an elastic member of the present invention shown in FIG. 14.

FIG. 16 is a sectional view of another embodiment of an elastic member of the present invention.

FIG. 17 is a sectional view of a compliant valve embodiment of the present invention located between the aorta and IVC.

FIG. 18 is a sectional view of a compliant valve embodiment of the present invention located within the heart chamber wall.

FIG. 19 is a sectional view of a compliant diaphragm embodiment of the present invention located within the hear chamber wall.

### DETAILED DESCRIPTION OF THE INVENTION

Stent with Internal Absorber

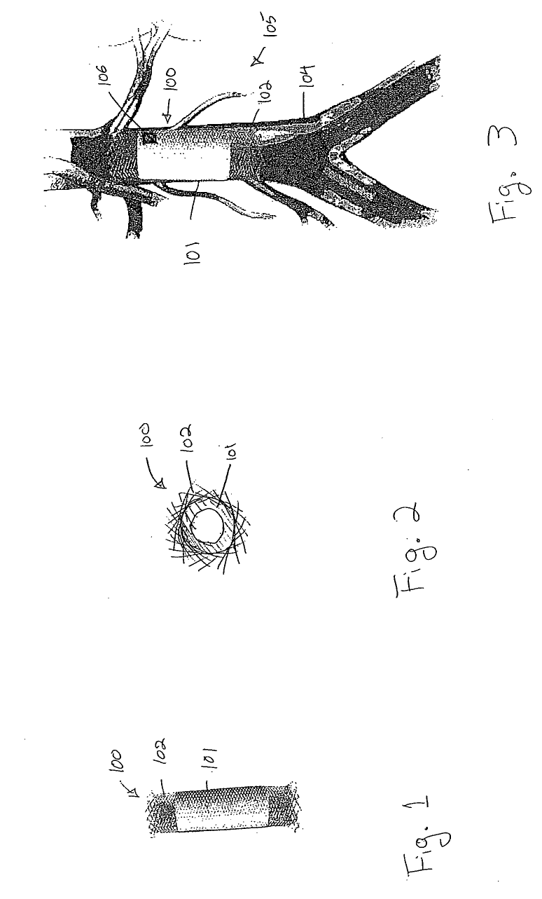

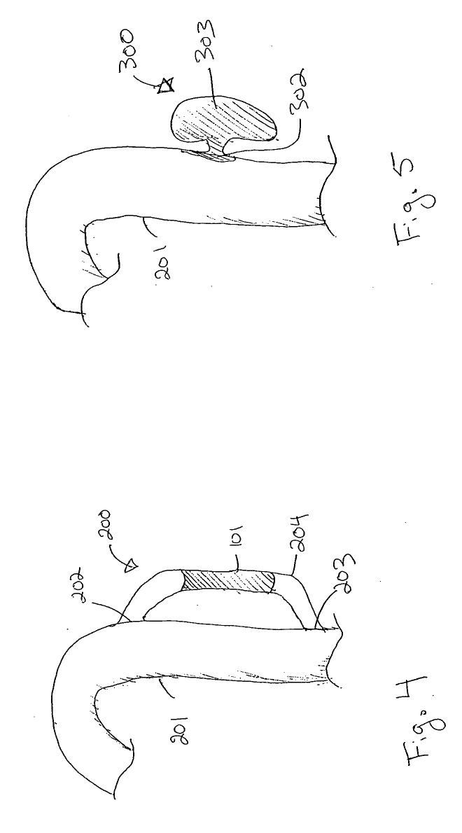

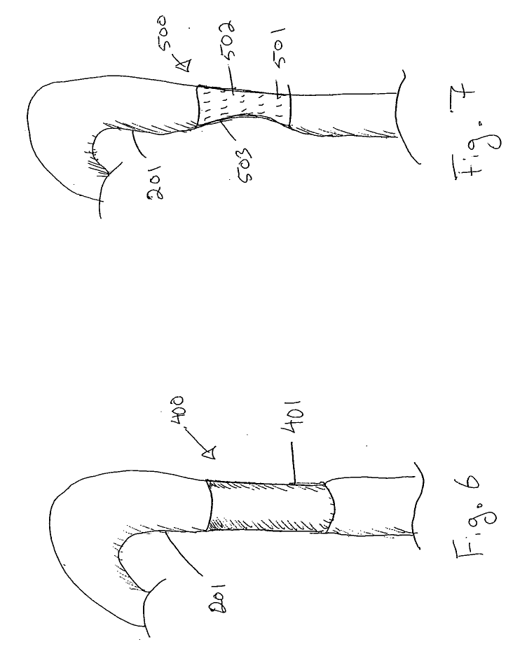

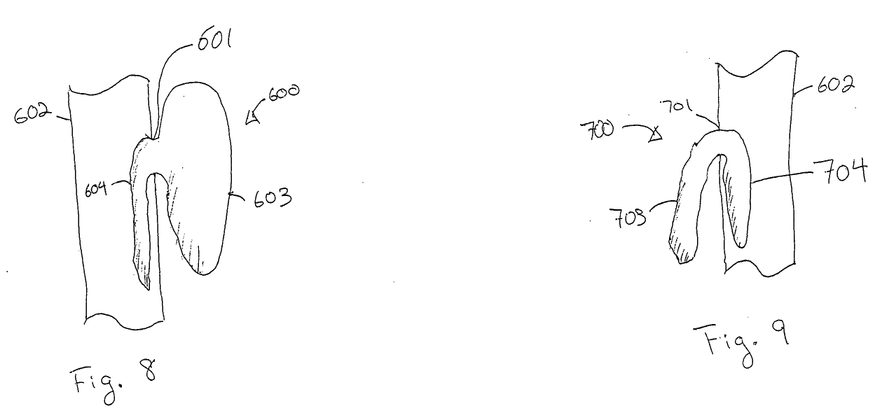

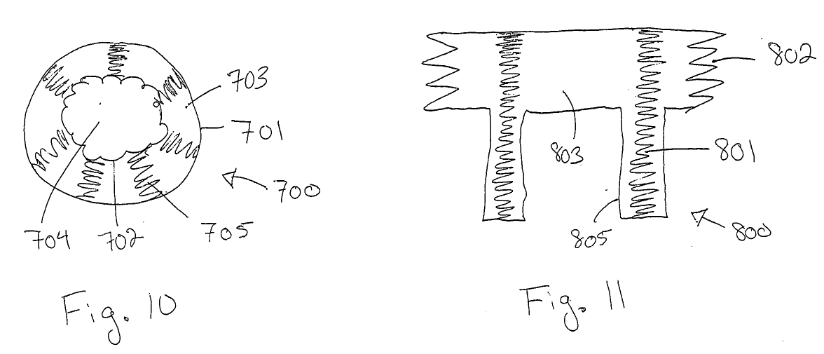

Referring to FIGS. 1-3, a body lumen compliance device 100 in accordance with one preferred embodiment of the present invention includes an anchoring structure 102 such as a stent, that has an open passage 103 therethrough. Mounted on the anchoring structure is an elastic member 101 that is positioned along at least a portion of the length of the anchoring structure 102.

In the embodiment shown, the elastic member 101 is shorter than the anchoring structure 102, thus leaving two exposed ends of the structure 102. The exposed ends can be used for enhancing the ability of the structure 102 to secure the entire device 100 at its desired location.

In the embodiment shown, the elastic member 101 is disposed internal to the anchoring structure 102. However, the elastic member 101 could be disposed on an external surface of the anchoring structure or could be made so as to be integrally woven within the anchoring structure 102. Either approach is acceptable so long as the elastic member provides the necessary elastic function to the device as described in greater detail below.

Referring to FIG. 3, a preferred site for use of the body lumen compliance device 100 in accordance with the present invention is in the descending aorta 104 of a patient having hypertension. In this regard, the body lumen compliance device 100 is situated such that the anchoring device 102 secures the device 100 against the internal walls of the descending aorta. The body lumen compliance device 100 is secured in place so as to eliminate migration but the elastic member 101 is positioned so as to provide the full extent of its elastic properties.

As will be understood by one of ordinary skill in the art, the pressure peaks encountered during the normal heart cycle by the aorta can be summarized as follows:

[image]

As such, in cases where the aorta has lost its compliance, the pressure peaks exert undue stress on the heart and particularly the left ventricle, requiring more energy, decreasing cardiac efficiency, as discussed above.

This system may also find use in cases of heart failure, where the heart pumps blood into the aorta in an inefficient manner. This may be caused by elimination of the vascular compliance through aortic stiffening. Such compliance elimination corresponds to an impedance mismatch, yielding energy wastage in an already failing heart. Restoration of the compliance, even in cases of normal blood pressure, will render the heart more efficient, and represent a therapy for heart failure.

In accordance with this embodiment of the invention, blood that is pumped into the aorta by the left ventricle is directed through the open passage 103 of the body lumen compliance device 100 and into the region of the device that includes the elastic member 101. As pressure increases from the pumping of the left ventricle beyond a desired pressure suitable for the patient, the elastic member 101 then absorbs this greater pressure by expanding its volume, so as to dilute the stress otherwise caused to the heart. In this fashion the elastic member 101 operates in a manner similar to a healthy aorta insofar as it “complies” or expands, and damps the pressure spikes caused by normal heart pumping and thus over time, greatly reduces the negative stress that is exerted on the heart muscle.

Parallel Compliance Device

Referring to FIG. 4, in accordance with another preferred embodiment of the present invention, a parallel body lumen compliance device 200 includes parallel compliance structure 204, having an open passage (not shown) therethrough. Each end of the parallel body lumen compliance device 200 secures to a vessel 201, allowing the open passage to fluidly connect to the interior of vessel 201 through device entrance 202 and device exit 203. Positioned along a portion of the length of the parallel body lumen compliance device 200 is an elastic member 101.

Parallel compliance structure 204 may be composed of an elastic membrane capable of providing the necessary structure to the parallel body lumen compliance device 200 and further sealing off the device from the body lumen as to prevent blood loss from the vascular system. It should be understood by one of ordinary skill in the art that a variety of materials, especially surgical or prosthetic vascular materials, may be used for the parallel compliance structure 204 providing they allow for blood containment and to maintain the device structure.

In the embodiment shown, an elastic member 101 is secured to a center region of the interior passage of parallel body lumen compliance device 200. Alternatively, the elastic member 101 may occupy a smaller region or stretch the entire length of parallel compliance structure 204.

This preferred embodiment shows elastic member 101 as secured within the passage, internal to the parallel body lumen compliance device 200. Alternatively, elastic member 101 could be disposed on the external surface of parallel compliance structure 204 or integrated together with compliance structure 204. Any of these approaches are acceptable provided they allow for necessary elastic function to the device as described further below.

In operation, blood is pumped into the vessel 201 and directed into device entrance 202. Blood passes through the passage of parallel body lumen compliance device 200 and back into the vessel 201 through device exit 203. As a spike of blood pressure pulses through the vessel 201, parallel body lumen compliance device 200 redirects a portion of the blood volume passing by device entrance 202 allowing elastic member 101 to absorb the pressure increase so as to decrease the stress otherwise caused to the heart. In this manner, the elastic member 101 mimics the operation of a healthy vessel in that it complies and dampens pressure spikes caused by a normal heart pumping.

Single Entry Compliance Device

Referring to FIG. 5, a single entry compliance device 300 includes a single entry compliance structure 301 having an internal cavity and an elastic member 303 positioned along a portion of the single entry compliance structure 301. Vessel opening 302 fluidly connects the interior of single entry compliance device 300 with the interior of vessel 201.

In this alternative preferred embodiment, single entry compliance structure 301 may be composed of an elastic membrane capable of providing the necessary structure to single entry compliance device 300 and further sealing off the device from the body lumen as to prevent blood loss from the vascular system. It should be understood by one of ordinary skill in the art that a variety of materials, especially surgical materials, may be used for the single entry compliance structure 301 providing they allow for blood containment and to maintain the device structure.

In this embodiment, the elastic member 303 is disposed onto the inner surface of single entry compliance structure 301. However, the elastic member 303 could be disposed on an external surface of single entry compliance structure 301 or integrated into the structure's surface. Either approach is acceptable so long as the elastic member provides the necessary elastic function to the device as described below.

Referring to FIG. 5, a preferred position for the use of the single entry compliance device 300 is proximate a vessel 201, more preferably in the descending aorta of a patient having a hypertension condition. Such positioning allows vessel opening 302 to secure to vessel 201 while providing an open passage from the interior of vessel 201 to the interior cavity of single entry compliance device 300.

In operation, blood is pumped into the vessel and directed through vessel opening 302 into the interior of the single entry compliance device 300 that includes elastic member 301. As pressure increases from the pumping of the heart beyond a desired pressure suitable for the patient, the elastic member 301 then absorbs this greater pressure so as to reduce the stress otherwise inflicted upon the heart. The cardiovascular system of the patient is thus able to function similar to that of a healthy patient, complying with and reducing spikes in pressure cause by normal heart pumping.

Outer Cuff-Like Compliance Device

Yet another preferred embodiment can be seen in FIG. 6. A compliant outer cuff device 400 is shown having a structural band 401 and an elastic member 101 (not shown).

When in a closed state, compliant outer cuff device 400 has an inner diameter being slightly smaller than the outer diameter of a desired vessel location. Therefore, the compliant outer cuff device 400 is secured around the outer diameter of a vessel 201, slightly compressing the original vessel diameter. The compliant outer cuff device 400 may have a number of mechanical devices for fastening the cuff around the vessel 201, such as clasps, hooks, or other securing devices, allowing for easy attachment to a desired location.

The elastic member 101 may be disposed on the inside surface of the structural band 401, as well as the outer surface, or even interwoven into the structural band 401. Any of these approaches will be acceptable so long as the elastic member 101 provides the necessary elastic function to the device.

In the embodiment shown, blood is pumped through the vessel 201, further passing through the region slightly compressed by the compliant outer cuff device 400. As pressure and volume increases in the compressed region of vessel 201, compliant outer cuff device 400 expands, acting to absorb this greater pressure. In this manner, the device acts to dilute and damp the natural pressure spikes caused by the heart.

Percutaneous Grabbing Compliant Device

In yet another preferred embodiment shown in FIG. 7, a grabbing compliant device 500 includes an anchor structure 503 having grabbing hooks 501 disposed about the outer surface of the structure and a passageway throughout. Integrated with the anchor structure 503 is an elastic member 502.

In the present embodiment, the elastic member 502 is interwoven with the anchor structure 503. The elastic member 502 may also be disposed on the inner or outer surface of the anchor structure 503. Either of these approaches may be acceptable provided they allow for the necessary elastic function described below.

The outer diameter of grabbing compliant device 500 may be slightly smaller than the inner diameter of the vessel 201. Such sizing allows the grabbing compliant device 500 to be percutaneously placed into a vessel 201 at a desired location. Grabbing hooks 501 covering the outer surface of the grabbing compliant device 500 are secured to the inner wall of the vessel 201, allowing for inward compression of the vessel 201 around the device.

In accordance with this embodiment of the invention, blood is pumped into vessel 201 by the heart, being directed through the compressed vessel region containing the grabbing compliant device 500. As blood pressure increases beyond a desired initial threshold, the elastic member 502 expands, momentarily increasing the diameter of the grabbing compliant device 500 and thus the diameter of the vessel 201. In this manner, the elastic member 502 acts to absorb this pressure spike, mimicking the compliance of a healthy vessel and greatly reducing the stress induced from normal heart pumping.

Internal/External Compliance Chamber

FIGS. 8 and 9 refer to two similar preferred embodiments of the present invention, illustrating internal/external compliant devices being located both internal to and external to the aorta or other vessel.

In FIG. 8, an internal/external balloon compliance device 600 includes an inner chamber 604 and outer chamber 603. The inner chamber 604 is tubular in shape, but other geometries may be employed so long as blocking of the blood flow in the aorta 602 is avoided.

Inner chamber 604 and outer chamber 603 form a single, continuous membrane having an inner cavity. Internal/external balloon compliance device 600 is positioned through the aorta wall 602 at an aorta wall entry hole 601, which is sealed around the device to prevent leakage of blood from the aorta while serving to hold the device in place.

FIG. 9 illustrates a similar embodiment as a tubular internal/external compliance device 700. Instead of a rounded, spherical shape, the outer chamber 703 conforms to a tubular, cylindrical shape. This cylindrical outer chamber 703 takes up less room outside the aorta or other vessel, but otherwise may possesses the same characteristics and benefits as the preferred embodiment of FIG. 8. Further, these embodiments provide the advantage of avoiding issues of working against absolute pressure instead of relative pressure.

In an alternative preferred embodiment, the compliance device is integral into a vascular graft, allowing for vascular repair as well as the ability to limit hypertension.

According to the present preferred embodiment, the internal cavity of the internal/external balloon compliance device 600 may be about 20-25 ml of volume inside the aorta and about 50-500 ml of volume outside the aorta. Varying volume amounts may be used, so long as the volume of the inner chamber does not block a significant portion of blood pumped through the aorta, the volume of the outer chamber does not interfere with organs external to the aorta, and the volume allows the device to provide a desired amount of elasticity as described below.

In the embodiments of FIGS. 8 and 9, desired elasticity is caused by adjusting the devices to pressure of about 40 mmHg, so as to cause about 10-55 ml of fluid or gas to run in and out of the aorta with each heartbeat. Additionally, the 10-55 ml of fluid flow is allowed to pass to the outer chamber 603 within about 0.1 seconds. Such flow time may best accomplished by using a gas, but a liquid may also be used. An additional port or valve opening may also be added to the chamber to allow adjustment of the chamber volume or pressure, as well as determine a threshold pressure to begin working.

In accordance with this embodiment of the invention, the outer membrane of the internal/external balloon compliance device 600 is composed of elastic biocompatible material, such as silicone or urethane. Portions of the device may also be composed of noncompliant material, so long as the overall desired compliance of the device is achieved.

The device may be coated with a biocompatible configuration, such as a microporus structure that encourages cell ingrowth, and endothelialization, with a cellular tissue surface integral as a result.

Referring to FIG. 8, blood is pumped into the aorta by the left ventricle and is directed past the inner chamber 604 of the internal/external balloon compliance device 600. As pressure increases from the pumping of the left ventricle beyond a desired pressure point, the inner chamber 604 compresses by pushing gas into outer chamber 603, thus absorbing the momentarily increased pressure that would otherwise cause stress to the cardiovascular system. In this manner, internal/external balloon compliance device 600 provides characteristics similar to a healthy aorta and represents a way of achieving the desired compliance in at least the embodiments of FIGS. 1-9.

Pressure Sensitive Valve Device

FIG. 17 shows a further embodiment of the present invention. A compliant valve device 1301 is composed of a pressure sensitive valve 1305 secured within passageway 1304.

In one preferred embodiment, compliant valve device 1301 is located between the aorta 1303 and the Inferior Vena Cava (IVC) 1302. Passageway 1304 secures to the aorta 1303 and IVC 1302, creating a passage to the interior of each. Pressure sensitive valve 1305 interrupts passageway 1304 preventing blood flow from passing through.

As blood pressure increases in the aorta 1303, the pressure sensitive valve 1305 opens at a predetermined level of blood pressure, allowing a small volume of blood to pass through to the IVC 1302. This redirection of a portion of blood reduces blood volume, further reducing the pressure. As the pressure in the aorta 1303 falls, the pressure sensitive valve 1305 closes. Thus, for the cost of a small volume of blood, about 20 ml, compliance is returned to the vascular system.

A variety of different surgical valves are known to one skilled in the art and may be used for pressure sensitive valve 1305, providing it allows for the above described properties.

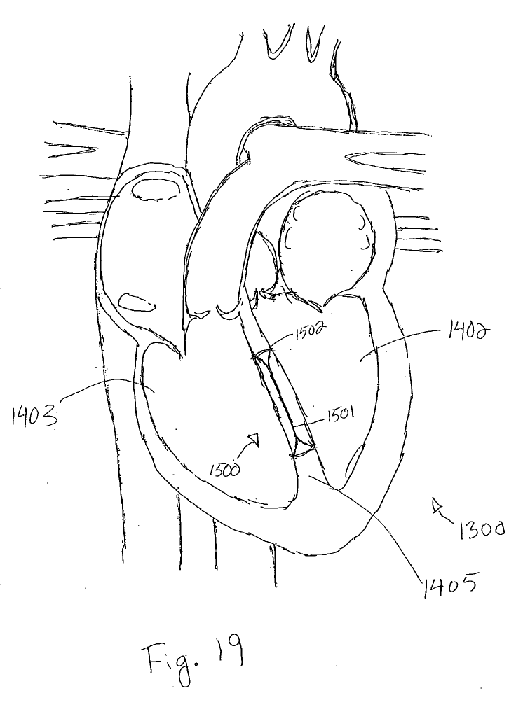

FIG. 18 shows an alternate position of a compliant valve device 1400 located in the heart chamber wall 1405 separating the right heart chamber 1403 from the left heart chamber 1402. Passageway 1401 is integrated into the heart chamber wall 1405, forming a passage between the two chambers. Pressure sensitive valve 1404 is secured within passageway 1401, preventing blood flow from passing through. Or, in the alternate, the pressure sensitive valve 1404 is simply inserted into the heart chamber wall.

When the blood pressure in the left ventricle increases during a heart beat, the pressure sensitive valve opens a predetermined level, allowing for a small volume of blood to pass from the left heart chamber 1402 to the right heart chamber 1403. As the pressure in the left heart chamber decreases, the valve closes, preventing blood flow between chambers. Thus, for the price of about 20 ml of blood redirection, compliance may be restored to a vascular system.

In one preferred embodiment of the present invention, the valve device 1305 and pressure sensitive valve 1404 can be based on known valve technology, e.g., a duckbill valve concept, a pressure relief valve concept, etc.

In another preferred embodiment, these valve devices can be based on a venturi valve concept so as to limit the danger of clotting. With the venturi valve, the valve is always open thus decreasing the potential for the blood to come to rest on a structure and thus causing a clot.

In yet another preferred embodiment, the valve devices could be based on a feedback control loop. For example, the valve could be actuated according to an electronic signal that is determined based on diagnostic measurements of the patient's condition. For example, an algorithm in a control module would evaluate such parameters as a patient's blood pressure, heart rate, body temperature, etc. and then arrive at a signal that opens or closes the valve in a manner that best addresses those parameters.

FIG. 19 illustrates yet another preferred embodiment of the present invention. A compliant diaphragm 1500 is composed of anchoring device 1502 and distensible membrane 1501.

The compliant diaphragm 1500 is preferably located in heart chamber wall 1405, between the left heart chamber 1402 and the right heart chamber 1403. Anchoring device 1502 secures distensible membrane 1501 within a sealed passage through the wall. Distensible membrane 1501 is composed of a pliable, distensible, biocompatible material, capable of stretching without breaking when pressure is applied. A variety of materials are available and known to a person of ordinary skill in the art to achieve the desired stretching functionality.

Unlike the previous compliant valve embodiment, blood does not pass between chambers of the heart. Instead, pressure increases in the left heart chamber 1402 as the heart 1300 begins to beat. As the pressure reaches a predetermined amount, the distensible membrane 1501 is pushed into the right heart chamber 1403, effectively increasing the volume of the left heart chamber. This volume increase serves to reduce peak blood pressure, restoring compliance and reducing stress and damage to the vascular system.

In this fashion, the pressure pikes of the blood flow caused by the beating of the heart are dampened by the above compliant device embodiments, allowing a patient's vascular system to approximate a more normal compliant function.

Both the compliant valve device 1301 and the compliant diaphragm 1500 may be used in tandem with other embodiments of the present invention, including the embodiments illustrated in FIGS. 1-9.

Vacuum Chamber with Spring Loading

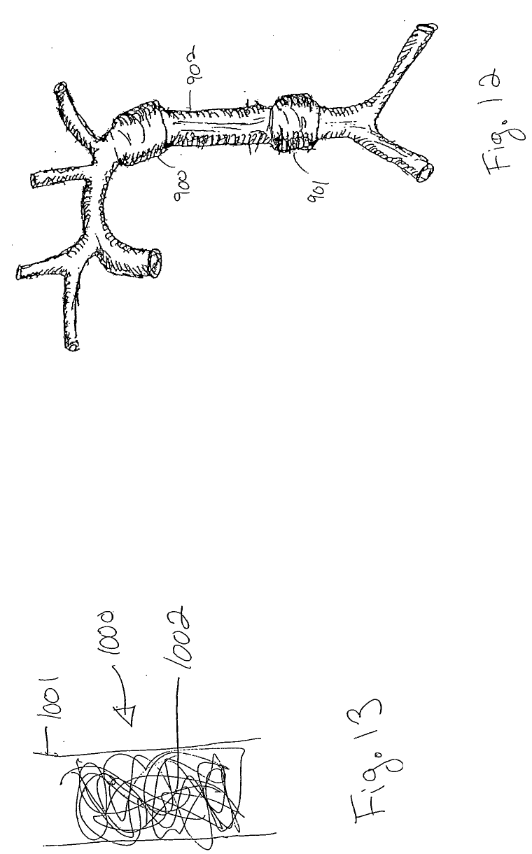

FIG. 10 illustrates yet another preferred embodiment of the present invention. This embodiment also describes the method of achieving compliance and can be used as the elastic member with at least the embodiments of FIGS. 1-9.

A vacuum chamber compliance device 700 is composed of rigid wall 701 and elastic wall 702, sealed together to form an internal cavity 703 and a central open passage 704 throughout.

In the present preferred embodiment, internal cavity 703 is vacuum sealed, the gas having been initially partially or completely removed. The internal cavity 703 is primarily held open by support springs 705 which may be composed of a variety of thermoplastic metals such as nitinol. Such thermoplastic metals allow the support springs 705 to be variably compliant and externally programmable by way of an external heat source, as described in further detail below. By carefully adjusting the support springs 705, a desired compliance may be obtained. Alternatively, the chamber may be loaded with a predetermined amount of gas, providing a further compliance variable.

The vacuum chamber compliance device 700 is anchored to the interior of an aorta or other vessel by way of the outer non compliant wall 701. The internal elastic wall 702 provides a compliant, elastic membrane capable of stretching with increased pressure against the support springs 705.

According to this preferred embodiment of the present invention, blood is pumped into the aorta by the left ventricle and is directed through the central open passage 704 of vacuum chamber compliance device 700. As blood pressure increases beyond a desired threshold, the springs compress to increase the internal diameter of the device, absorbing the blood pressure spike. This absorption of shock mimics the compliance of a healthy cardiovascular system, decreasing overall stress.

Referring to FIG. 11, an alternate preferred embodiment of the spring loaded vacuum chamber is also presented as a pillar vacuum chamber compliance device 800, including an elastic membrane 805 sealed around support springs 801 extending away from the device body. The inner cavity 803 of the device is sealed, forming bellows 802 on the body side.

Internal cavity 803 is vacuum sealed, the gas having been initially removed to form a partial or near-complete vacuum. The internal cavity 803 is primarily held open by support springs 801 which may be composed of a variety of thermoplastic metals such as nitinol. Such thermoplastic metals allow the support springs 801 to be variably compliant and externally programmable by way of an external heat source as described below. By carefully adjusting the support springs 801, a desired compliance may be obtained.

Pillar vacuum chamber compliance device 800 is placed percutaneously into an aorta or other vessel.

The pillar vacuum chamber compliance device 800 operates in a similar fashion to the device of FIG. 10, in that blood is pumped into the aorta by the left ventricle and is directed past the body of the device. As blood pressure increases beyond a desired threshold, the springs compress to decrease the body size of the device, absorbing the blood pressure spike. This absorption of shock mimics the compliance of a healthy cardiovascular system, decreasing overall stress.

Multiple Compliance Devices

A further aspect of the present invention allows for the utilization of multiple compliance devices strategically placed at desired locations of the cardiovascular system. By utilizing multiple compliance devices, the overall compliance of a patient's cardiovascular system can be further adjusted to mimic that of a young healthy system.

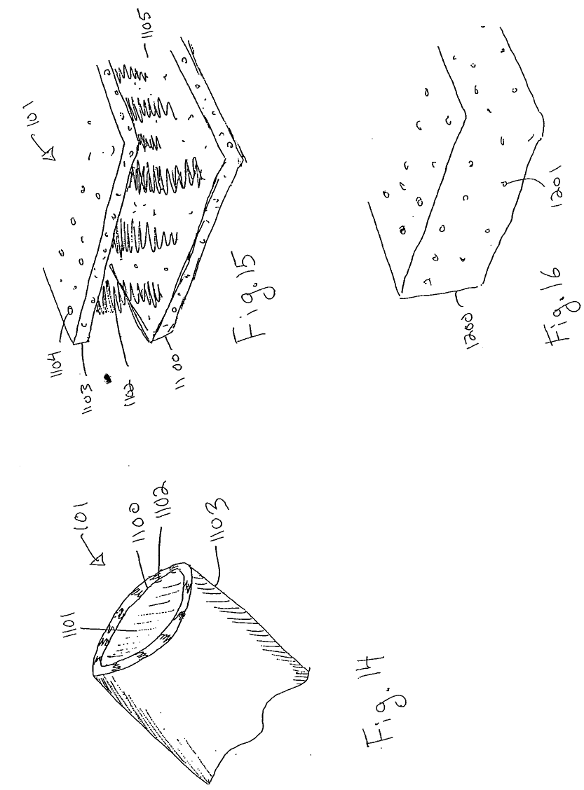

FIG. 12 illustrates such usage of the present invention in an aorta 902 having a first compliant device 900 and a second compliant device 901 positioned in a lower area of aorta 902. Any of the previously mentioned embodiments of the present invention may be used in such a multiple compliant system so long as they function with the overall desired compliancy necessary to reduce blood pressure spike induced stress.

Sandwiched Springs

As seen above, many of the aforementioned preferred embodiments make use of an elastic member to provide underlying elasticity and pliability, thus allowing the devices that use such an elastic member to be compliant within a vascular system.

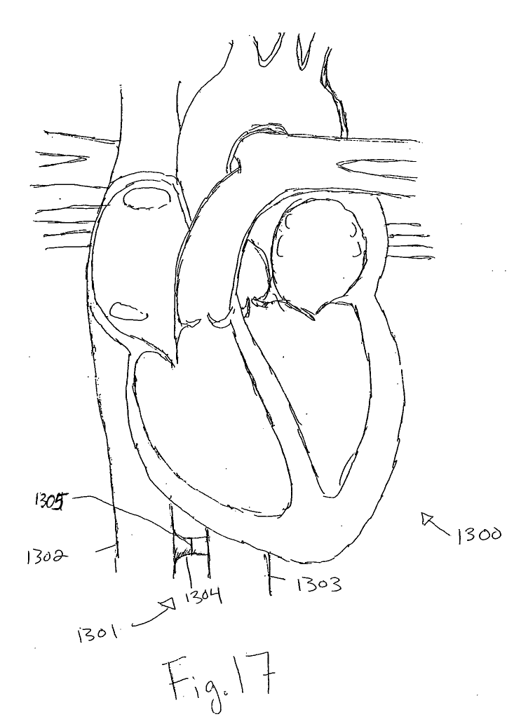

One such preferred embodiment of an elastic member can be seen in FIGS. 14 and 15. Elastic member 101 includes an inner elastic membrane 1100, forming an inner passage 1101. Outer elastic membrane 1103 seals to the edges of inner elastic membrane 1100, forming an inner cavity 1105 containing springs 1102.

In the embodiment shown, the inner elastic membrane 1100 and outer elastic membrane 1103 are composed of an elastic, biocompatible material allowing the device to expand and contract as needed. Additionally, these elastic membranes contain bio-pores 1104 for cellular in-growth, allowing the device to become one with the patient. Such in-growth is an important consideration to the long-term health and survival of the compliance system in the patient. Preferred pore size varies from about 20 to 200 microns and the bio-pores 1104 may further connect through the aortic or vessel wall in addition to interconnecting with each other to maximize cellular in-growth. Optionally, the compliant vascular device may posses inflammation inducing properties for fibrosis stimulation which, in connection with the bio-pores 1104, serve to further adhere the device to the vessel walls through in-growth of fibrous tissue.

In the present preferred embodiment, springs 1102 are fixed to the inner elastic membrane 1100 and outer elastic membrane 1103, spanning the space inside inner cavity 1105. Inner cavity 1105 may further contain a gas, liquid, or a vacuum to further modify the compliance of device as discussed further below.

Springs 1102 are preferably composed of a thermo-plastic metal such as nitinol. These materials allow the elastic member 101 to be variably compliant and externally programmable through the use of a carefully directed heat source. The springs may be heated transcutaneously with a number of different energy types, such as radio frequency or ultra sonic energy. As the springs 1102 are heated, their spring constants change, depending on the properties of the material used.

In addition to changing the overall compliance of the springs 1102, the pressure induced compliance threshold may be modified. This value represents the minimum amount of pressure required for the device to act in a compliant fashion. Increasing the spring constant of the springs 1102 increases the threshold, while decreasing the spring constant reduces the threshold. Thus, the thermo-plastic metal of the springs 1102 allow a physician to adjust the slope of compression, linearity/spring function shape, cut points, and regulation threshold. The pressure within the chamber may be externally modified by adding or subtracting material from the chamber.

The maximum preferred volume of the compliant vascular device is about 30 ml, or about the volume that might easily fit into the descending thoracic aorta. The preferred compliance volume is about 10-55 ml, meaning that the compliant vascular device will change in volume by about that amount within a tenth of a second from the natural aortic pressure change, typically about 90 mmHg to 130 mmHg. The spring should be carefully adjusted to stretch a desired amount over a pressure range. Such adjustments may be made in accordance with Hooke's law which states that a spring will stretch over its elastic range roughly in proportion to the tension or compression applied to it. Therefore, the geometry of the chamber and spring may be such that about a 5% or 10% elongation of the spring causes a 100% change in the volume of the chamber.

Unitary Elastic Member

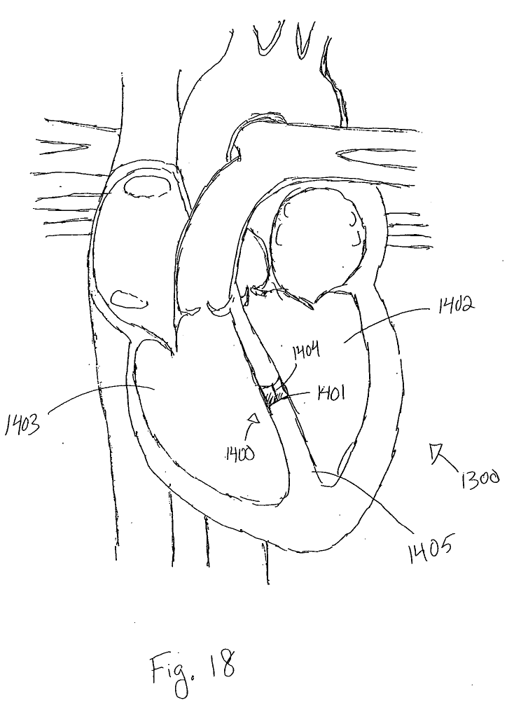

Referring to FIG. 16, an unitary elastic member 1200 is composed of a solid elastic material such as silicone, other plastic polymers, rubbers, nitinol meshes, polyurethanes, and other similar elastic materials.

An alternative preferred embodiment (not shown) of the unitary elastic member 1200 includes springs completely embedded within the elastic material. These springs may be pre-programmed for a desired compliance before incorporation into the elastic material.

Filamentous Network Elastic Member

FIG. 13 illustrates yet another preferred embodiment of the present invention. A filamentous network elastic member 1000 includes a plurality of elastic filaments 1002, sealed within an elastic membrane 1001.

The elastic filaments 1002 are composed of an elastic material or springs enclosed in a pliable material, allowing the structure to compress and reduce volume. By further arranging multiple elastic filaments 1002 together in a radial configuration, the filamentous network compliant device 1000 efficiently acts to absorb pressure shock.

As the blood pressure increases, the elastic membrane 1001 pushes against the elastic filaments 1002. This pressure causes the elastic filaments 1002 to not only compress closer to each other, but themselves compress in size. Thus, as blood pressure increases above a certain level, the filamentous network elastic member 1000 reduces in size, decreasing blood pressure and reducing the stress associated with this increased pressure.

Such an embodiment of an elastic member may be used in connection with any types of compliant devices, including those described in this invention, so long as they allow for adequate placement of the elastic member to absorb a desired amount of blood pressure.

Biasing Substance

The above mentioned preferred embodiments of the compliant vascular devices may have a media bias in the elastic member 101, as seen in FIG. 1, or a media bias in the internal cavity, as seen in FIG. 8. By modifying the media bias of a compliant device, the overall compliance, and therefore the overall performance of the device may be modified. Such media may include gas, such as nitrogen or carbon dioxide, a liquid, such as water or blood, or lack of material such as a partial or complete vacuum.

An inner cavity such as inner cavity 1105 in FIG. 15 or chamber 603 in FIG. 8 may be accessed externally and filled or emptied of media, modifying compliance of the device. A preferred embodiment includes a connection to the subcutaneous tissues that is accessible by needle procedure subcutaneously and into a conduit that communicates with the compliance chamber. The injected material may also have a chemical process that changes a chemical composition within the chamber to alter compliance. These methods of adjusting the media bias not only permit alteration of compliance, but also maintaining proper compliance as the system ages.

Pressure Spikes

The above mentioned preferred embodiments of the compliant vascular devices may need to equalize air pressure/atmospheric pressure to take best advantage of optimal dynamic range. An air or gas based connection will bias the offset on the chamber's elasticity to that of the ambient pressure in the body, reflecting external pressure. A preferred embodiment for such venting includes one or more venting spikes that connect from an inner chamber of the compliance device to outside the aorta, such as the peritoneal cavity or thorax. The connections may be in the form of spikes containing a lumen, and may push through the aorta into the surrounding cavity. The devices may be prong-like in configuration and extend radially outward from the support device to safely puncture the aortic wall.

Transducer

Any of the embodiments of the present invention may also include a transducer 106 capable of telemetry outside the body, as seen in FIG. 3. The transducer may measure such values as device volume, flow outside the device, device pressure (inside), and the pressure outside the device. Thus, this compliant vascular device will permit measurement of the phase angle between any or all of these parameters and permit appropriate adjustment of parameters to effect a positive hemodynamic change. The pressure, volume, flow, and velocity information may be used in a feed back loop to alter the device pressure-volume relationship for optimal cardiovascular system effects. Such effects may be lessening of ventricular work, altering phase angles between pressure, velocity, flow, lowering pressure or raising pressure. The device may also possess programmable pressure-volume relationships from either external or internal features. This may involve heating, cooling, or magnetic means to alter the compliance.

The compliant vascular device may be implanted in a variety of locations in the body such as the aorta or other vascular vessels. Further, the device may be placed at renal arteries to increase apparent pressure at the kidneys. There is an apparent wave reflection point induced at the renal arteries to stimulate a blood pressure reduction. The apparent pressure increase is induced out-of-phase with volume/flow to limit the systemic effects of the apparent pressure. This will induce compensatory renal feedback mechanisms to lower systemic pressure through natural mechanisms.

The device may also be placed in the carotid arteries to alter local hemodynamics (pressure dynamics) at the crotid sinuses providing specific biologic feedback.

AAA Repair with Specific Shock Absorber Compliance

The compliant vascular device described in the embodiments above may also be used for aneurysm repair (thoracic, abdominal, abdominal aortic, or elsewhere), particularly in connection with abdominal aortic aneurysm repair (AAA) such as shown in U.S. Pat. No. 6,344,052, which is herein incorporated by reference. The standard AAA graft material is made expansile to absorb stroke volume from the heart and create a re-phasing shift to compliance, lowering systolic blood pressure. The device may or may not be throughout the entire length of the system, including the iliac limbs of the system. This generates greater lengths for volume absorption. The device may stretch down into the iliac bifurcation and beyond into the iliac portions of the graft to have a large volume of absorption and limit the required distance of expansion. Multifilar support may be included, with different filar supports having differential expansion constants. The device may also have great dynamic range, to prevent fatigue. The covering of the device may be elastic/expansile as well to allow expansion. There is an external, protective covering to serve as a safety layer that prevents rupture by overexpansion as may occur in later stages of the graft device.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A device for absorbing fluid pressure in a body lumen comprising:
an elastic member having a passage therein; and
an anchoring member sized and shaped to secure said elastic member to a body lumen; said elastic member having an elasticity selected so as to provide a dampening response to increases in fluid pressure in said body lumen.

2. The device according to claim 1 wherein said anchoring member is sized and shaped so as to secure said elastic member to an inside of a said body lumen.

3. The device according to claim 2 wherein said anchoring member is a stent.

4. The device according to claim 2 wherein said anchoring member is secured to an outer circumference of said elastic member.

5. The device according to claim 1 wherein said anchoring member is sized and shaped so as to secure said elastic member to an outside surface of said body lumen.

6. The device according to claim 5 wherein said elastic member is sized and shaped to encircle an external surface of said body lumen.

7. The device according to claim 5 wherein said anchoring member encircles said elastic member, thereby securing said elastic member to said body lumen.

8. The device according to claim 6 wherein said elastic member has an inner diameter which is smaller than an outer diameter of the body lumen.

9. The device according to claim 1 wherein said anchoring member and said elastic member are integral.

10. The device according to claim 1 wherein said anchoring member comprises a hollow tubular structure having a first end and a second end; said first and second ends sized for attachment to said body lumen so as to be in fluid communication with said body lumen.

11. The device according to claim 1 wherein said elastic member is comprised of a hollow membrane structure having multiple springs.

12. The device according to claim 11 wherein said springs are composed of a thermo-plastic metal.

13. The device according to claim 12 wherein said thermo-plastic metal is nitinol.

14. The device according to claim 1 wherein said elastic member is comprised of a hollow membrane structure containing a plurality of elastic filaments.

15. The device according to claim 1 wherein said elastic member contains pores between about 20 microns and about 200 microns in size.

16. The device according to claim 1 wherein said elastic member substantially resides outside said body lumen.

17. The device according to claim 16 wherein said elastic member substantially resides outside said body lumen.

18. A method of absorbing fluid pressure in a body lumen of a patient comprising:
diagnosing a chronic elevated lumen pressure condition in said patient;
inserting an implant in said body lumen of said patient; and
allowing flow of body lumen fluid into said implant, absorbing at least a portion of said elevated lumen pressure condition with said implant.

19. A method according to claim 18 wherein diagnosing a chronic elevated lumen pressure includes diagnosing hypertension.

20. A method according to claim 18 wherein the absorbing with said implant includes allowing said implant to change internal diameters in response to said elevated lumen pressure condition.

21. A method according to claim 18 wherein the absorbing with said implant includes dampening said elevated lumen pressure conditions with a plurality of springs internal to said implant.

22. A method according to claim 18 wherein the absorbing with said implant includes dampening said elevated lumen pressure conditions with a gaseous media within said implant.

23. A method according to claim 18 wherein said implant further comprises an elastic member having an inner chamber containing a plurality of elastic filaments.
